(19) United States
(12) Patent Application Publication
(10) Pub. No.: US 2010/0130444 A1
(43) Pub. Date: May 27, 2010

(54) COMPLEXES OF PROSTAGLANDIN DERIVATIVES AND MONOSUBSTITUTED, CHARGED BETA-CYCLODEXTRINS

(76) Inventors: El Mustapha Belgsir, Poitiers (FR); Randell Gatz, Alpnach Dorf (CH); Yves Cenatiempo, Saint Julian rars (FR); Frederic Turpin, Tours (FR)

Correspondence Address: Richard Voellmy, Av de Sully 67, La Tour-de-Peilz, 1814, CH

(21) Appl. No.: 12/452,491

(22) PCT Filed: Jul. 3, 2008
(86) PCT No.: PCT/IB2008/001783

§ 371 Date: Jan. 5, 2010

Related U.S. Application Data
(60) Provisional application No. 60/929,763, filed on Jul. 11, 2007.

(51) Int. Cl. A61K 31/724; C08B 30/18; A61P 27/02

(52) U.S. Cl. 514/58; 536/46

(57) ABSTRACT

The present invention relates to water-soluble, non-covalent complexes of a group of prostaglandin derivatives including latanoprost and monosubstituted, charged β-cyclodextrins, as well as uses of these complexes in therapeutic compositions that are administered topically for treating intraocular hypertension and glaucoma.

### FIELD OF THE INVENTION

The present invention relates to novel complexes consisting of a prostaglandin derivative and a mono-substituted β-cyclodextrin, and their use in therapeutic compositions and in therapy of elevated intraocular pressure and glaucoma.

### BACKGROUND OF THE INVENTION

Glaucoma is caused by elevated relative intraocular pressure that results in optic nerve damage and visual field loss. Lee and Higginbotham (2005) Am. J. Health-Syst. Pharm. 62, 691-699. If untreated, this disease causes irreversible blindness. Glaucoma is a common disease. In the United States alone, more than two million people are believed to suffer from glaucoma, and over 80'000 residents are legally blind as a result of the disease. The prevalence of glaucoma is particularly elevated in the elderly, African Americans and patients with diabetes, hypertension and myopia. Glaucoma comprises a group of different diseases, of which primary open-angle glaucoma is the most common type. Treatment of glaucoma focuses on reducing increased intraocular pressure by pharmacological or surgical means. Intraocular pressure in the range of 10-21 mm Hg is considered normal. First-line pharmacological treatment is typically employing a topical selective or non-selective beta-adrenergic antagonist or a topical prostaglandin derivative. Prostaglandins reduce intraocular pressure by increasing aqueous humor outflow in the eye. Prostaglandin derivatives were developed and are currently used for therapy that are similarly potent as natural prostaglandins but essentially devoid of the limiting property of natural prostaglandins of causing superficial irritation and vasodilation in the conjunctiva. U.S. Pat. Nos. 4,599,353, 5,296,504, 5,422,368, 6,429,226, 5,510,383, 5,688,819, 6,403,649. Several drugs based on the latter prostaglandin derivatives are on the market; they have proven to be safe and to cause relatively few systemic adverse effects (e.g., Xalatan, Travatan, Lumigan). The main disadvantages of these drugs are increased iris pigmentation, hypertrichosis of eyelashes, intraocular inflammation and sensations of burning, itching and stinging. The present invention relates to improved formulations comprising prostaglandin derivatives for the topical treatment of intraocular hypertension and glaucoma. The preferred prostaglandin derivatives, representatives of which are the active substances in several approved medications, have low aqueous solubility. Consequently, the presently marketed formulations include compounds aiding solubilization such as polyoxyl 40 hydrogenated castor oil in the case of Travatan or benzalkonium chloride in the case of Xalatan, which compounds are known to cause discomfort to some patients, i.e., stinging, burning and itching eyes. Hence, at least with aqueous formulations, it has been difficult to reach a pharmaceutically effective dose because of the limited solubility of the drug substance, without resorting to addition of irritating solubility-enhancing agents. Furthermore, it has not been possible to prepare solid dosage forms of prostaglandin derivatives that readily dissolve in an aqueous opthalmologically compatible vehicle. Moreover, as the preferred prostaglandin derivatives are uncharged at pH values that are compatible with topical ophthalmic uses, they do not exhibit an affinity for the charged cornea and are rapidly washed away by the tear fluid. The present invention relates to opthalmologic compositions comprising a non-covalent complex of selected prostaglandin derivatives and monosubstituted, positively charged β-cyclodextrins. These complexes are stable over extended periods of time and are highly soluble in aqueous vehicles, permitting administration of drug substance at any desired concentration up to about 15 mg/ml without the use of an irritating solubility enhancer. Lyophilized complexes dissolve instantaneously in aqueous solutions and, therefore, are suitable for use in solid dosage forms. Moreover, the complexes are positively charged at pH values at which opthalmological formulations are typically used, which feature is expected to increase the residence time of the drug substance on the corneal surface and, consequently, its absorption by the cornea. Finally, prostaglandins are generally unstable; by contrast, prostaglandins in complexes of the invention are stabilized. The use of pure monosubstituted cyclodextrins rather than mixtures of cyclodextrins that are substituted to various degrees in the pharmaceutical compositions of the present invention enables their accurate and unambiguous chemical description.

### SUMMARY OF THE INVENTION

The present invention relates to a water-soluble, non-covalent complex of (a) a derivative of a prostaglandin (PG) having the general structure

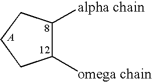

wherein A represents the alicyclic ring C₈-C₁₂ of PGA, PGB, PGD, PGE or PGF; the alpha chain has the structure

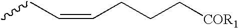

wherein R₁ is an alkyloxy or alkylamino group, preferably with 1-10 carbons, especially 1-6 carbons; and
   the omega chain is defined by the formula

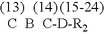

wherein B is a single bond or a double bond, C is a carbon atom, the number being indicated within parentheses, D is a carbon chain with 2-5, especially 3 carbon atoms, C₁₅ having a carbonyl or (S)—OH substituent and C₁₆-C₁₉ having lower alkyl substituents, or preferably H, and R₂ is a phenyl ring optionally having substituents selected among alkyl, alkoxy and fluorocarbon groups;
   and (b) a monosubstituted derivative of β-cyclodextrin having the structure

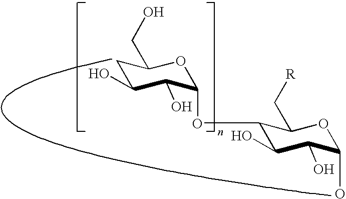

wherein n equals 6 and R is —NH₂⁺—(CH₂)ₚ—OH or —NH₂⁺—(CH₂)ₚ—NH₃⁺ (at neutral to acidic pH), p being an integer from 2-6.

In preferred complexes of the invention, B in the omega chain of the prostaglandin is a single bond, D is a chain of 3 carbon atoms, and R₂ is a phenyl group. In more preferred complexes of the invention, the derivative of a prostaglandin is either 15-dehydro-17-phenyl-18,19,20-trinor-PGF_{2α}-isopropylester, 13,14-dihydro-17-phenyl-18,19,20-trinor-PGA₂-isopropylester, 15-(R)-17-phenyl-18,19,20-trinor-PGF_{2α}, latanoprost, bimatoprost or travoprost. In still more preferred complexes, the derivative of a prostaglandin is either latanoprost, bimatoprost or travoprost, and in the most preferred complexes it is latanoprost. Preferred derivatives of β-cyclodextrin are those in which the C₆ substituent R is either NH₂⁺—(CH₂)₃—NH₃⁺ or —NH₂⁺—(CH₂)₃—OH. In preferred complexes of the invention, the derivative of a cyclodextrin and the derivative of a prostaglandin are present at a molar ratio from 1:1 to 30:1, and in the most preferred complexes at a ratio of 5:1 to 10:1.

The invention also relates to therapeutic compositions for topical treatment of ocular hypertension and glaucoma comprising one of the aforementioned water-soluble, non-covalent complexes of a derivative of a cyclodextrin and a derivative of a prostaglandin, and an opthalmologically compatible vehicle, wherein the prostaglandin is present in an effective amount, which is an amount sufficient to reduce intraocular pressure. In preferred complexes of the invention utilized in therapeutic compositions, B in the omega chain of the prostaglandin is a single bond, D is a chain of 3 carbon atoms, and R₂ is a phenyl group. In more preferred complexes, the derivative of a prostaglandin is either 15-dehydro-17-phenyl-18,19,20-trinor-PGF_{2α}-isopropylester,13,14-dihydro-17-phenyl-18,19,20-trinor-PGA₂-isopropylester,15-(R)-17-phenyl-18,19,20-trinor-PGF_{2α}, latanoprost, bimatoprost or travoprost. In still more preferred complexes, the derivative of a prostaglandin is latanoprost, bimatoprost or travoprost, and in the most preferred complexes it is latanoprost. Preferred derivatives of β-cyclodextrin are those in which the C₆ substituent (R) is either —NH₂⁺—(CH₂)₃—NH₃⁺ or —NH₂⁺—(CH₂)₃—OH. In preferred complexes of the invention utilized for the preparation of therapeutic compositions, the derivative of a cyclodextrin and the derivative of a prostaglandin are present at a molar ratio from 1:1 to 30:1 and in the most preferred complexes at a ratio of 5:1 to 10:1. The opthalmologically compatible vehicle is an aqueous solution that may contain one or more opthalmologically acceptable salts, an isotonic agent and a buffer or other pH-controlling agent. In particular embodiments, therapeutic compositions may also include a viscosity-increasing agent, a non-irritating preservative or an anti-oxidant.

The invention also relates to containers for dispensing a therapeutic composition of the invention in a drop-wise fashion to an eye of a patient. In a particular embodiment, such a container is partitioned into two or more compartments, of which one compartment comprises a water-soluble, non-covalent complex of the invention in a dry form and another compartment comprises an opthalmologically compatible vehicle, and includes means for inducing mixing of the contents of said two compartments such that a therapeutic composition of the invention is constituted. Also encompassed by the invention are kits containing two or more of said containers. In a related embodiment, the invention concerns kits comprising a first container that comprises a water-soluble, non-covalent complex of the invention in a dry form and another container that comprises an opthalmologically compatible vehicle.

A further embodiment of the invention relates to a method of treating glaucoma or intraocular hypertension in an eye of a patient, comprising topical administration to the eye of the patient of a therapeutic composition of the invention.

### BRIEF DESCRIPTION OF THE DRAWING

FIG. 1. Phase solubility diagram of the latanoprost-mono-6-desoxy-6-diaminopropyl-β-cyclodextrin system in water and at room temperature. BCD A56 is mono-6-desoxy-6-diaminopropyl-β-cyclodextrin.

### DETAILED DESCRIPTION OF THE INVENTION

The present invention relates to non-covalent complexes comprising a member of a group of prostaglandin derivatives and a member of a group of monosubstituted β-cyclodextrins, therapeutic compositions comprising such a complex for the topical treatment of elevated intraocular pressure and glaucoma and the use of such therapeutic compositions for the treatment of elevated intraocular pressure and glaucoma. The non-covalent complex of the invention can be prepared by dissolving a mono-substituted β-cyclodextrin of the invention in water at a concentration of, typically, 20-100 mM. To this solution an appropriate amount of a prostaglandin derivative of the invention is added, and the suspension is stirred in the dark for a period of, preferably, 24 h to 72 h at a temperature between ambient temperature and 70° C. A temperature close to ambient temperature is preferred. An appropriate amount of a prostaglandin derivative is an amount that preferably yields a molar ratio of cyclodextrin to prostaglandin of between 1:1 and 30:1, and most preferably between 5:1 and 10:1. The resulting solution that may contain small amounts of undissolved prostaglandin derivative is subjected to centrifugation and is then passed through a 0.45 μm filter. This step removes any undissolved prostaglandin. Finally, solid complex is recovered by lyophilization.

The prostaglandin derivatives that can be included in a complex of the invention are of the following general structure

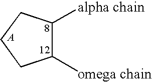

wherein A represents the alicyclic ring C₈-C₁₂ of PGA, PGB, PGD, PGE or PGF; the alpha chain has the structure

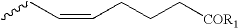

wherein R₁ is an alkyloxy or alkylamino group, preferably with 1-10 carbons, especially 1-6 carbons; and
   the omega chain is defined by the formula

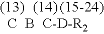

wherein B is a single bond or a double bond, C is a carbon atom, the number being indicated within parentheses, D is a carbon chain with 2-5, especially 3 carbon atoms, C₁₅ having a carbonyl or (S)—OH substituent and C₁₆-C₁₉ having lower alkyl substituents, or preferably H, and R₂ is a phenyl ring optionally having substituents selected among alkyl, alkoxy and fluorocarbon groups.
   Preferred prostaglandin derivatives are

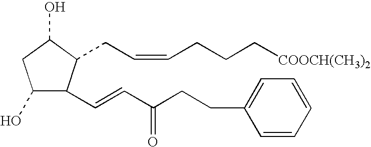

15-dehydro-17-phenyl-18,19,20-trinor-PGF_{2α}-isopropylester,

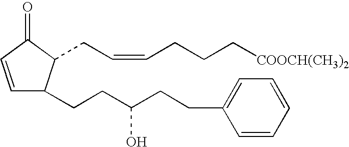

13,14-dihydro-17-phenyl-18,19,20-trinor-PGA₂-isopropylester,

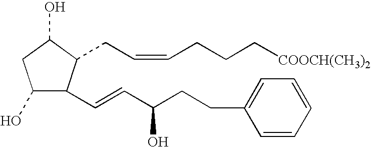

15-(R)-17-phenyl-18,19,20-trinor-PGF_{2α}-isopropylester,

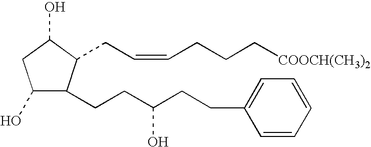

13,14-dihydro-17-phenyl-18,19,20-trinor-PGF_{2α}-isopropylester also known as latanoprost,

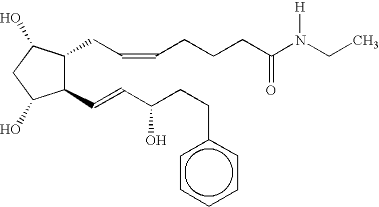

17-phenyl-18,19,20-trinor-PGF_{2α}-ethylamide also known as bimatoprost, and

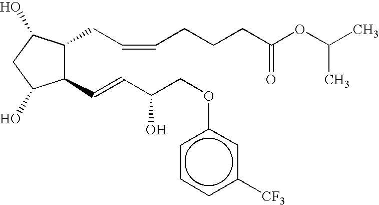

16-(3-trifluoromethylphenoxy)-17,18,19,20-tetranor-PGF_{2α}-isopropylester also known as travoprost.

The most preferred prostaglandin derivative is latanoprost. The prostaglandin derivatives of the invention and methods for their synthesis are described in U.S. Pat. Nos. 5,422,368, 5,510,383, 5,688,819 and 6,403,649, which patents are incorporated herein by reference in their entirety, or are generally known in the art. Several of the prostaglandin derivatives of the invention including latanoprost, bimatoprost and travoprost can be obtained from Cayman Chemical Company, Ann Arbor, Mich.

The mono-substituted β-cyclodextrins that can be included in a complex of the invention are of the following general structure

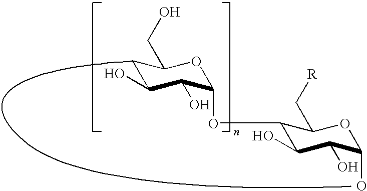

wherein n equals 6 and R is —NH₂⁺—(CH₂)ₚ—OH or —NH₂⁺—(CH₂)ₚ—NH₃⁺ (at acidic pH), p being an integer from 2-6. These monosubstituted cyclodextrins and methods for their synthesis were described in international patent application PCT/FR94/01501 and U.S. Pat. No. 5,760,017, both incorporated herein in their entirety by reference. The cyclodextrins can be obtained from BioCyclex SAS, Poitiers, France.

Therapeutic compositions for topical administration to the eye of a patient can be prepared by dissolving an amount of a complex consisting of an effective amount of a prostaglandin derivative and a cyclodextrin derivative of the invention in an opthalmologically compatible vehicle. An effective amount of a prostaglandin derivative of the invention in such a therapeutic composition is an amount that is capable of causing a reduction in intraocular pressure in a patient and of maintaining such reduced pressure over time when administered regularly. Preferably, such effective amount is an amount that causes a reduction in intraocular pressure between about 15% and 30% relative to the pressure measured prior to therapy or that returns intraocular pressure to a value within the normal range of 10-21 mm Hg. Methods for measuring intraocular pressure are well known in the art. Goldman tonometry is probably the most widely used method. An effective amount of a prostaglandin derivative is between 0.1 μg and 30 μg per eye and topical administration, topical administration typically occurring at a frequency of not more than once or twice a day. Preferably, the effective amount is between 1 μg and 10 μg of prostaglandin derivative.

An opthalmologically compatible vehicle suitable for use with complexes of the invention consisting of a prostaglandin derivative and a cyclodextrin derivative is an aqueous solution that may contain one or more opthalmologically acceptable salts, an isotonic agent and a buffer or other pH-controlling agent. An opthalmologically acceptable salt is any salt that does not diminish the activity of the topical therapeutic compositions of the invention and that does not impart any deleterious or untoward effects on the eyes of the patient to which it is administered as part of the pharmaceutical compositions and that has no negative systemic effects. The opthalmologically compatible vehicle may further include an isotonic agent and a buffer or other pH-controlling agent. These excipients may be added for the attainment of preferred ranges of pH (about 3.5-8.0) and osmolarity (from about 260 to 320 mosm/L). Examples of suitable buffers are acetate, borate, carbonate, citrate and phosphate buffer. Such buffers may be present in a therapeutic composition in concentrations from 0.01 to 1.0% (w/v). An isotonic agent may be selected from any of those known in the art, e.g. mannitol, dextrose, glucose and sodium chloride, or other electrolytes. Preferably, the isotonic agent is glucose or sodium chloride. The isotonic agents may be used in amounts that impart to the pharmaceutical composition the same or essentially the same osmotic pressure as tear fluid. The concentration of isotonic agent in the aqueous solution will depend upon the nature of the particular isotonic agent used and may range from about 0.1 to 10%. When glucose is used it is preferably used in a concentration of from 1 to 5% w/v, more particularly 5% w/v. When the isotonic agent is sodium chloride, it is preferably employed in amounts of up to 1% w/v, in particular 0.9% w/v.

Especially when formulated for multiple uses, therapeutic compositions of the invention may further contain a non-irritating preservative. Examples of opthalmologically compatible preservatives are triamino-dipropylene cocoylamide, triamino-dipropylene oleylamide, polyhexamethylene biguanidine, stabilized oxychloro complexes (such as those known as PuriteR), phenylmercuric acetate, chlorobutanol, sorbic acid, chlorhexidine, benzyl alcohol, parabens, and thimerosal. Typically, such preservatives are present at concentrations from about 0.001 to 1.0%. When a therapeutic composition contains a preservative, it may also include an appropriate chelating agent such as, for example, a salt of edetate.

Therapeutic compositions of the invention may also include a viscosity-increasing or thickening agent. Preferred thickening agents are cellulose and cellulose-derivative thickening agents such as alkyl celluloses and hydroxyalkyl celluloses. Examples for this type of thickening agent are methyl cellulose and hydroxypropyl methylcellulose (e.g., Nos. 2208 or 2906 as defined in the Japanese and U.S. Pharmacopeias). Other thickening agents include carboxyvinyl polymers, polyvinyl polymers and polyvinylpyrrolidones. Examples of polyvinyl polymers are polyvinyl acetates and polyvinyl alcohols, and example polyvinylpyrrolidones are poly-N-vinylpyrrolidones and vinylpyrrolidone co-polymers.

The therapeutic compositions of the invention may further comprise an anti-oxidant. Opthalmologically acceptable anti-oxidants include sodium metabisulfite, sodium thiosulfate, acetylcysteine, butylated hydroxyanisole, and butylated hydroxytoluene. An oxidant should be present at a concentration below that at which it causes irritation of the eye. Typically, the concentration of an anti-oxidant should be within the range from about 0.0001 to about 0.01% (w/v) to preclude eye irritation. Alternatively, the composition may be stored under nitrogen and, optionally, in the presence of a free oxygen scavenger (for example, Fe).

Therapeutic compositions of the invention may be dispensed as drops from a container suitable for such a purpose. Such a container is any container that is suitable for dispensing individual drops that are of a size appropriate for opthalmologic use. A suitable container may also be a container holding a single dose of a therapeutic composition of the invention and that is capable of dispensing this dose, as one or more drops, during a single administration to one or both eyes of a patient. Alternatively, a suitable container may have at least two compartments, one comprising dry non-covalent complex of the invention and the other comprising an opthalmologically compatible vehicle, the two compartments being allowed to communicate at an opportune time resulting in the constitution of a therapeutic composition of the invention. The therapeutic composition of the invention may also be provided in the form of a kit that comprises two or more such containers. An alternative kit for providing a therapeutic composition of the invention may comprise a container comprising dry non-covalent complex of the invention and another container comprising an opthalmologically compatible vehicle.

The therapeutic compositions of the invention can be used for the treatment of intraocular hypertension and glaucoma. Typically, a patient will be administered daily one or two drops of the therapeutic composition (corresponding to a volume of about 30 μl) in one or both eyes as needed. The amount of prostaglandin derivative delivered per eye will be from 0.1 to 30 μg, preferably from 1 to 10 μg. Typically, the treating physician will observe the patient and will also determine the effectiveness of the drug regimen in reducing intraocular pressure. Based on these observations, dosage and frequency of administration may be optimized. A therapeutic composition of the invention may be used as a single drug or in combination with other anti-glaucoma drugs. For example, a treatment regimen may combine a composition of the invention and a suitable α-adrenergic agonist or a topical or systemic carbonic anhydrase inhibitor. Combination of a composition of the invention with a β-adrenergic antagonist such as timolol may also be found to enhance therapeutic efficacy.

The invention is further elaborated by the following examples. The examples are provided for purposes of illustration to a person skilled in the art and are not intended to be limiting the scope of the invention as described in the claims. Thus, the invention should not be construed as being limited to the examples provided, but should be construed to encompass any and all variations that become evident as a result of the teaching provided herein.

### EXAMPLES

### Example 1

### Solubilization of Latanoprost

A library of unsubstituted and monosubstituted cyclodextrins was screened to identify those cyclodextrin derivatives that have the highest capacity for solubilization of latanoprost. Complexation was carried out in ultra-pure water at a molar ratio of cyclodextrin derivative to latanoprost of 5:1, corresponding to concentrations of 115.58 mM and 23.12 mM for cyclodextrin derivative and latanoprost, respectively. Because of the known sensitivity of latanoprost to light, high temperature and oxidation, experiments were conducted in the dark and under a nitrogen atmosphere at room temperature. Selective results are shown in Table 1. It was found that use of β-cyclodextrins containing on a single C₆ position of a glucose unit a substituent of the type —NH₂⁺—(CH₂)ₚ—NH₃⁺ or —NH₂⁺—(CH₂)ₚ—OH (p from 2 to 6) resulted in a dramatic increase in aqueous solubility of latanoprost as exemplified by mono-6-desoxy-6-diaminopropyl-β-cyclodextrin and mono-6-desoxy-6-aminopropanol-β-cyclodextrin. Non-covalent complexes formed contained the cyclodextrin derivative and latanoprost at a molar ratio of 5:1. No other type of monosubstituted cyclodextrin tested augmented the aqueous solubility of latanoprost to a comparable degree.

TABLE 1
Solubilization of Latanoprost

| | Mass Ratio | | Maximal | |
|---|---|---|---|---|
| | Cyclodextrin:Latanoprost | Molar Ratio | Solubility of | Solubility |
| Cyclodextrin | in | Cyclodextrin:Latanoprost | Latanoprost | Increase |
| Derivative | Complex | in Complex | (mg/ml) | (S/S₀) |
| No addition | N/A | N/A | 0.045 (Exp. S₀) | N/A |
| β-Cyclodextrin¹ | 59 | | 0.255 | 5.67 |
| α-Cyclodextrin² | >50 | | <0.200 | <4.44 |
| Mono-6-desoxy- | 14 | 5 | 9.57 | 213 |
| 6-diaminopropyl- | | | | |
| β-cyclodextrin³ | | | | |
| Mono-6-desoxy- | 14 | 5 | 10.2 | 227 |
| 6-aminopropanol- | | | | |
| β-cyclodextrin⁴ | | | | |
| Mono-6-desoxy- | 106 | 38 | 1.30 | 28.9 |
| 6- | | | | |
| methylthioureido- | | | | |
| β-cyclodextrin⁵ | | | | |
| Monoanhydro-β- | 65 | 25 | 2.00 | 44.4 |
| cyclodextrin⁶ | | | | |

¹β-cyclomaltoheptaose;
²α-cyclomaltooctaose;
³6¹-(3-amino-propylamino)-6¹-deoxycyclomaltoheptaose;
⁴6¹-(3-hydroxy-propylamino)-6¹-deoxycyclomaltoheptaose;
⁵6¹-(methylthioureido-6¹-deoxycyclomaltoheptaose;
⁶3¹,6¹-anhydrocyclomaltoheptaose

### Example 2

### Solubility of Latanoprost, Bimatoprost And Travoprost in 50 Mm Mono-6-desoxy-6-diaminopropyl-β-cyclodextrin, Mono-6-desoxy-6-aminopropanol-β-cyclodextrin or Water

TABLE 2
Solubilization of prostaglandin derivatives

| | | In 50 mM Mono-6- | In 50 mM Mono-6- |
|---|---|---|---|
| | | desoxy-6- | desoxy-6- |
| Prostaglandin | | diaminopropyl-β- | aminopropanol-β- |
| Derivative | In Water | cyclodextrin | cyclodextrin |
| Latanoprost (g/lt) | 0.089 | 5.90 ± 0.06 | 5.72 ± 0.08 |
| Bimatoprost (g/lt) | 3.019 | 10.68 ± 0.67 | 10.77 ± 0.05 |
| Travoprost (g/lt) | 0.032 | 4.94 ± 0.13 | 4.95 ± 0.02 |

### Example 3

### Characterization of Complexation: Latanoprost-Cyclodextrin Complex

The minimal time period required for reaching maximal solubility of the guest molecule, i.e., latanoprost, in a solution containing 115 mM mono-6-desoxy-6-diaminopropyl-β-cyclodextrin was determined. Subsequent to addition of latanoprost, the resulting suspension was ultrasonicated for 5 min and then stirred magnetically at room temperature and in the dark for a period of 48 hours. Aliquots were removed at 0, 1, 3, 6, 12, 24 and 48 hours. Each aliquot was filtered through a 0.45 um PVDF (Millipore/Whatman) membrane, and the filtrate was diluted for quantitative analysis of latanoprost by HPLC. Results indicated that solubility equilibrium was reached at about 24 hours. Subsequent experiments were conducted after 24 hours of equilibration.

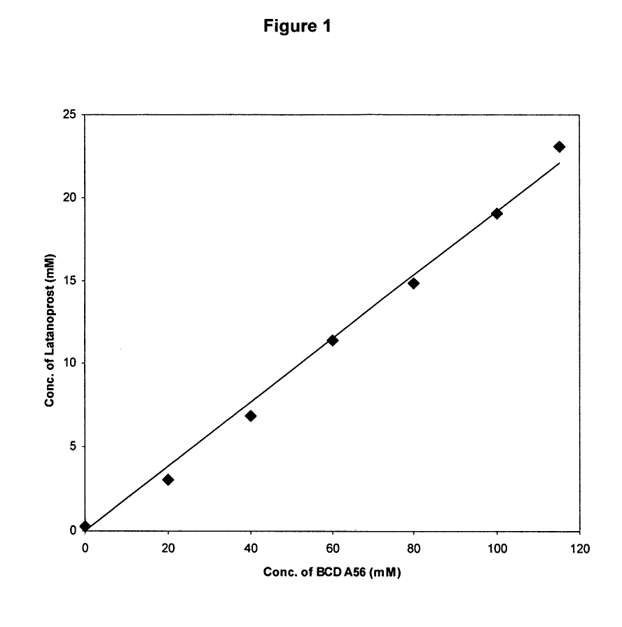

A phase solubility diagram was constructed to examine the solubility increase of latanoprost in the presence of increasing concentrations of mono-6-desoxy-6-diaminopropyl-β-cyclodextrin. The experiment was aimed at evaluating inclusion stoichiometry at various concentrations and to determine optimal molar ratios for the preparation of latanoprost-cyclodextrin derivative complexes (in water, and at room temperature). Results (FIG. 1) indicated that optimal ratios ranged from 5:1 at latanoprost concentrations greater than about 20 mM to about 8:1 at latanoprost concentrations below 2 mM.

### Example 4

### Short Term Stability of Latanoprost Cyclodextrin Complex

The aim of this experimentation was to define conditions under which aqueous solubility of latanoprost was insensitive to concentration and temperature. Latanoprost-mono-6-desoxy-6-diaminopropyl-β-cyclodextrin complexes in water were prepared containing cyclodextrin and latanoprost at molar ratios of 5:1, 6:1, 7:1 and 8:1, respectively. Subsequent to filtration, complexes were lyophilized to dryness. The different complexes were then dissolved in water to different concentrations and incubated either at room temperature or at 60° C. for a period of 7 days. Aliquots were taken at regular intervals, and were filtered, diluted and subjected to quantitative analysis of latanoprost by HPLC. Results indicated that there was a reduction of latanoprost concentration in the 5:1 and 6:1 complexes at elevated concentrations of latanoprost (close to 10 mg/ml), but not at low concentrations (50 μg/ml). In contrast, 7:1 and 8:1 complexes were stable over the entire period of observation at all concentrations and at both temperatures.

### Example 5

### Properties of Solid Latanoprost-Cyclodextrin Complex

Solid non-covalent complex of latanoprost and mono-6-desoxy-6-diaminopropyl-β-cyclodextrin was obtained by complexation in water as before. A solution of mono-6-desoxy-6-diaminopropyl-β-cyclodextrin was prepared in water. Latanoprost was added to obtain a 1:8 stoichiometry of latanoprost to cyclodextrin. This solution was stirred at room temperature and in the dark for 24 hours. After filtration, the clear solution was lyophilized to dryness. The resulting product was a flaky white powder of low density.

Aliquots of this complex were redissolved in water for analysis by quantitative HPLC. The basic properties of the complex as determined by HPLC are listed in Table 3.

TABLE 3
Latanoprost - Mono-6-desoxy-6-diaminopropyl-β-cyclodextrin Complex

| | |
|---|---|
| Molar Ratio (latanoprost:cyclodextrin derivative) | 1:8.3 |
| Mass Ratio | 1:23.1 |
| Solubility Limit of Complex | 370 mg/ml |
| Solubility Limit of Latanoprost | 16.1 mg/ml |

### Example 6

### Stability of Complexed Latanoprost

Latanoprost-mono-6-desoxy-6-diaminopropyl-β-cyclodextrin complex was prepared essentially as described under the previous example. Complex was dissolved in water to yield solutions (pH 6.7) containing 15 mg/ml and 7.5 mg/ml of latanoprost, respectively, or in a standard, isotonic phosphate buffer (pH 6.7) at 50 μg/ml. A control solution of 50 μg/ml latanoprost in phosphate buffer was also prepared. One-milliliter amounts of these solutions were transferred to pre-scored hermetically thermo-sealed ampoules, and the sealed ampoules were incubated at room temperature, at 37° C. or at 60° C., in the presence or absence of natural light, for a total of 30 days. Aliquots were removed periodically and were analyzed by HPLC using either a UV diode array detector (at 210 nm) or an evaporative light scattering detector (ELSD). Example results obtained using ELSD are shown in Table 4. Results indicate relative amounts of intact latanoprost left after 30 days of incubation as a percentage of the sum of intact latanoprost and degradation product (presumably, the free acid form of latanoprost). Complexed latanoprost, especially at the higher concentrations, was found to be stable under all conditions of temperature and light, whereas uncomplexed latanoprost (the control solution) exhibited instability, in particular at 25° C. Such instability had been observed previously. Morgan (2001) J. Glaucoma 10, 401-405; Yusuke Sakai (2005) Int. J. Pharmaceutics 305, 176-179. International patent publication WO 2004/024164.

Analogous experiments were carried out to estimate the stability of latanoprost-mono-6-desoxy-6-aminopropanol-β-cyclodextrin complex. Table 5 contains the results of such an experiment. As was observed in the previously described experiment, complexed latanoprost, especially at the higher concentrations, was found to be remarkably stable under all conditions of temperature and light. The discrepancy between complexed and uncomplexed latanoprost showed dramatically at 25° C., at which temperature there was very little degradation of complexed latanoprost, but abundant degradation of uncomplexed latanoprost.

TABLE 4
HPLC Analysis of Uncomplexed Latanoprost and Latanoprost
Complexed with Mono-6-desoxy-6-diaminopropyl-β-cyclodextrin
after 30 Days of Incubation

| | |
|---|---|
| Storage conditions | Relative area of |

| | | | |
|---|---|---|---|
| | Temp. | | Latanoprost (%) |
| Formulation | (° C.) | Exp. | after 30 days |

| | | | |
|---|---|---|---|
| 15 g/l Latanoprost | 25 | Light | 100 |
| solubilized with | | Dark | 100 |
| mono-6-desoxy-6- | 37 | Light | 100 |
| diaminopropyl-β- | | Dark | 100 |
| cyclodextrin in | 60 | Light | 99.0 |
| distilled water at pH | | Dark | 99.0 |
| 6.7 | | | |
| 7.5 g/l Latanoprost | 25 | Light | 99.9 |
| solubilized with | | Dark | 100 |
| mono-6-desoxy-6- | 37 | Light | 99.5 |
| diaminopropyl-β- | | Dark | 99.9 |
| cyclodextrin in | 60 | Light | 98.4 |
| distilled water at pH | | Dark | 98.7 |
| 6.7 | | | |
| 50 mg/l Latanoprost | 25 | Light | 100 |
| solubilized with | | Dark | 100 |
| mono-6-desoxy-6- | 37 | Light | 100 |
| diaminopropyl-β- | | Dark | 100 |
| cyclodextrin in a | 60 | Light | 93.0 |
| standard phosphate | | Dark | 98.8 |
| buffer isotonic | | | |
| Latanoprost in a | 25 | Light | 76.9 |
| standard phosphate | | Dark | 85.9 |
| buffer isotonic | 37 | Light | 99.8 |
| | | Dark | 100 |
| | 60 | Light | 94.7 |
| | | Dark | 98.5 |

Sample analysis and quantification were performed on a customized Dionex Summit Dual-Gradient HPLC system equipped with a P680 pump, an ASI-100 automated injector, a TCC-100 column oven, a UVD340U diode array detector, a Polymer laboratories evaporative light scattering detector (PL-ELS 2100), a Merck Chromolith ® Performance RP-18 endcapped (100-4.6 mm) column and a Merck Chromolith ® guard cartridge RP-18e (5-4.6 mm). The mobile phases were prepared from HPLC-grade acetonitrile and water acidified by TFA at concentration of 100 μl/l. The acetonitrile/water gradient was set as follows: from 10/90 to 90/10 in 10 min; stable for 2 min, then to 10/90 in 0.5 min; and finally stable for 2.5 min. Flow rate was 1 ml/min. The temperature of the column oven was set to 30° C. The injection volume was 20 μl. All chromatographic data management was ensured by the Chromeleon software in its 6.7 version (Dionex, U.S.A.).

TABLE 5
HPLC Quantitative Analysis of Uncomplexed Latanoprost and
Latanoprost Complexed with Mono-6-desoxy-6-aminopropanol-β-cyclodextrin
(“cyclodextrin”) after 30 Days of Incubation

| | | | | Latanoprost | Latanoprost | Degradation |
|---|---|---|---|---|---|---|
| Cyclodextrin | | | | in complex | in complex | products of |
| complex of | | | | at day 0 | at 30 days | latanoprost |
| latanoprost | | | | (g/lt) [or | (g/lt) [or | at 30 days |
| or aqueous | Latanoprost | | | latanoprost | latanoprost | (% of sum |
| solution of | concentration | Temperature | | in aqueous | in aqueous | of drug and |
| latanoprost | (g/lt) | (° C.) | Exposure | solution*] | solution*] | deg. prod.) |
| Cyclodextrin | 9 | 25 | Light | 8.77 +/− 0.399 | 8.64 +/− 0.329 | 0 |
| complex | | | | | | |
| Cyclodextrin | 9 | 25 | Dark | 8.77 +/− 0.399 | 8.64 +/− 0.307 | 0 |
| complex | | | | | | |
| Cyclodextrin | 9 | 37 | Light | 8.77 +/− 0.399 | 8.40 +/− 0.606 | 0 |
| complex | | | | | | |
| Cyclodextrin | 9 | 37 | Dark | 8.77 +/− 0.399 | 8.66 +/− 0.123 | 0 |
| complex | | | | | | |
| Cyclodextrin | 9 | 60 | Light | 8.77 +/− 0.399 | 8.66 +/− 0.791 | 0 |
| complex | | | | | | |
| Cyclodextrin | 9 | 60 | Dark | 8.77 +/− 0.399 | 8.59 +/− 0.149 | 0 |
| complex | | | | | | |
| Cyclodextrin | 1 | 25 | Light | 0.94 +/− 0.050 | 0.93 +/− 0.050 | 0 |
| complex | | | | | | |
| Cyclodextrin | 1 | 25 | Dark | 0.94 +/− 0.050 | 0.94 +/− 0.007 | 0 |
| complex | | | | | | |
| Cyclodextrin | 1 | 37 | Light | 0.94 +/− 0.050 | 0.91 +/− 0.552 | 0 |
| complex | | | | | | |
| Cyclodextrin | 1 | 37 | Dark | 0.94 +/− 0.050 | 0.94 +/− 0.087 | 0 |
| complex | | | | | | |
| Cyclodextrin | 1 | 60 | Light | 0.94 +/− 0.050 | 0.85 +/− 0.011 | 0 |
| complex | | | | | | |
| Cyclodextrin | 1 | 60 | Dark | 0.94 +/− 0.050 | 0.76 +/− 0.239 | 0 |
| complex | | | | | | |
| Cyclodextrin | 0.05 | 25 | Light | 0.050 +/− 0.004 | 0.029 +/− 0.007 | 0.63 |
| complex | | | | | | |
| Cyclodextrin | 0.05 | 25 | Dark | 0.050 +/− 0.004 | 0.013 +/− 0.009 | 0.24 |
| complex | | | | | | |
| Cyclodextrin | 0.05 | 37 | Light | 0.050 +/− 0.004 | 0.048 +/− 0.001 | 0 |
| complex | | | | | | |
| Cyclodextrin | 0.05 | 37 | Dark | 0.050 +/− 0.004 | 0.048 +/− 0.023^{#} | 0^{#} |
| complex | | | | | | |
| Cyclodextrin | 0.05 | 60 | Light | 0.050 +/− 0.004 | 0.027 +/− 0.001 | 3.40 |
| complex | | | | | | |
| Cyclodextrin | 0.05 | 60 | Dark | 0.050 +/− 0.004 | 0.025 +/− 0.010 | 2.40 |
| complex | | | | | | |
| Aqueous | | 25 | Light | 0.063 +/− 0.003 | 0.006 +/− 0.004 | 77.9 |
| solution | | | | | | |
| Aqueous | | 25 | Dark | 0.063 +/− 0.003 | 0.002 +/− 0.001 | 93.4 |
| solution | | | | | | |
| Aqueous | | 37 | Light | 0.063 +/− 0.003 | 0.061 +/− 0.003 | 0 |
| solution | | | | | | |
| Aqueous | | 37 | Dark | 0.063 +/− 0.003 | 0.051 +/− 0.001 | 0 |
| solution | | | | | | |
| Aqueous | | 60 | Light | 0.063 +/− 0.003 | 0.043 +/− 0.007 | 0 |
| solution | | | | | | |
| Aqueous | | 60 | Dark | 0.063 +/− 0.003 | 0.058 +/− 0.002 | 0 |
| solution | | | | | | |

Experiment performed in phosphate-citrate buffer at pH 5.0.
^{#}15-day measurements.

### Example 7

### Ocular Penetration and Activation of Latanoprost Subsequent to Administration of Latanoprost-Cyclodextrin Complex in Pigmented Rabbits

Thirty-six pigmented rabbits from the Fauve de Bourgogne strain (male, 2-2.5 kg) were randomly divided into four treatment groups of nine animals. Each group was subdivided into three subgroups of three animals corresponding to the three chosen time points of sacrifice after last administration of latanoprost formulation (15 min, 1 h and 2 hrs). The animals received in each eye 5 instillations of 50 microliter each of the tested formulations within a period of 20 min. After the above-indicated delays, animals were sacrificed. Aqueous humor was sampled and analysed for latanoprost and latanoprost acid form (activated form of latanoprost) using an LC/MS method. Test items were:
1. aqueous solution of latanoprost-mono-6-desoxy-6-aminopropanol-β-cyclodextrin complex (1:5 molar ratio) containing 600 microgram/ml of latanoprost
2. aqueous solution of latanoprost-mono-6-desoxy-6-aminopropanol-β-cyclodextrin complex (1:5 molar ratio) containing 200 microgram/ml of latanoprost
3. aqueous solution of latanoprost-mono-6-desoxy-6-aminopropanol-β-cyclodextrin complex (1:5 molar ratio) containing 20 microgram/ml of latanoprost
4. commercial aqueous formulation of latanoprost (Xalatan^{R}) containing 50 microgram/ml of latanoprost

Table 6 reports results of measurements of the activated form of latanoprost (acid) in aqueous humor, and Table 7 presents corresponding data for latanoprost.

TABLE 6
Content of Acid Form of Latanoprost (ng/ml of aqueous humor)

| Time | | | | |
|---|---|---|---|---|
| after | | | | |
| last | | | | |
| instil- | | | | |
| lation | | | | |
| (h) | Test item 1 | Test item 2 | Test item 3 | Test item 4 |
| 0.25 | 971 +/− 406 | 255 +/− 59.6 | 99.4 +/− 37.2 | 72.1 +/− 25.5 |
| 1 | 1510 +/− 582 | 509 +/− 139 | 212 +/− 48.7 | 162 +/− 39.3 |
| 2 | 1560 +/− 275 | 496 +/− 133 | 165 +/− 33.9 | 169 +/− 31.0 |

TABLE 7
Content of Latanoprost (ng/ml of aqueous humor)

| Time after last | | | | |
|---|---|---|---|---|
| instillation (h) | Test item 1 | Test item 2 | Test item 3 | Test item 4 |
| 0.25 | ND* | ND | ND | ND |
| 1 | ND | ND | ND | ND |
| 2 | ND | ND | ND | ND |

*ND, not detected.
Detection limit was 10 ng/ml of vitreous humor.

The data presented in Table 6 indicate that latanoprost administered in the form of latanoprost-mono-6-desoxy-6-aminopropanol-β-cyclodextrin complex penetrates the eye at least 2.5 times better than the commercial aqueous formulation. As comparison with the data from Table 7 reveals, latanoprost delivered from the cyclodextrin complex is essentially completely converted into the active form during or subsequent to penetration. These results suggest that a therapeutically effective intraocular concentration of latanoprost can be reached with a significantly reduced administered dose of latanoprost, provided that the latanoprost is administered as a cyclodextrin complex of the invention, raising the expectation of reduced external side effects due to latanoprost. Alternatively, if this were desired, latanoprost-cyclodextrin complex of the invention may be administered at elevated concentrations to achieve intraocular concentrations of latanoprost acid that are many fold higher than those attained upon administration of the commercial aqueous formulation.

### Example 8

### Typical Therapeutic Compositions for Topical Administration Useful for Treating Elevated Intraocular Pressure and Glaucoma

| Component | % w/v |
|---|---|
| Prostaglandin derivative of the invention | 0.002-0.06 |
| Cyclodextrin derivative of the invention | 0.0046-1.38 |
| NaOH/HCl | q.s. pH 3.5-8.0 |
| Purified water | q.s. 100 ml |

### Example 9

### Typical Therapeutic Compositions Comprising Latanoprost Useful for Topical Treatment of Elevated Intraocular Pressure and Glaucoma

| Component | % w/v |
|---|---|
| Latanoprost | 0.002-0.06 |
| Mono-6-desoxy-6-aminopropanol-β- | 0.0046-1.38 |
| cyclodextrin | |
| NaOH/HCl | q.s. pH 3.5-8.0 |
| Purified water | q.s. 100 ml |

### Example 10

### A Preferred Therapeutic Composition Useful for Topical Treatment of Elevated Intraocular Pressure and Glaucoma

| Component | % w/v |
|---|---|
| Latanoprost | 0.005 |
| Mono-6-desoxy-6-aminopropanol-β- | 0.115 |
| cyclodextrin | |
| NaOH/HCl | q.s. pH 5.5 |
| Purified water | q.s. 100 ml |

### Example 11

### A Further Preferred Therapeutic Composition Useful for Topical Treatment of Elevated Intraocular Pressure and Glaucoma

| Component | % w/v |
|---|---|
| Latanoprost | 0.005 |
| Mono-6-desoxy-6-aminopropanol-β- | 0.115 |
| cyclodextrin | |
| Polyhexamethylene biguanidine | 0.005 |
| Edetate disodium | 0.01 |
| Sodium chloride | 0.9 |
| NaOH/HCl | p.s. pH 5.5 |
| Purified water | q.s. 100 ml |

### Example 12

### A Further Preferred Therapeutic Composition Useful for Topical Treatment of Elevated Intraocular Pressure and Glaucoma

| Component | % w/v |
|---|---|
| Latanoprost | 0.005 |
| Mono-6-desoxy-6-diaminopropyl-β- | 0.115 |
| cyclodextrin | |
| Polyhexamethylene biguanidine | 0.005 |
| Sodium chloride | 0.9 |
| Hydroxypropyl methylcellulose | 0.5 |
| Edetate disodium | 0.01 |
| NaOH/HCl | p.s. pH 5.5 |
| Purified water | q.s. 100 ml |

1. A non-covalent complex of
(a) a derivative of a prostaglandin having the general structure 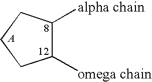
wherein A represents the alicyclic ring C₈-C₁₂ of PGA, PGB, PGD, PGE or PGF; the alpha chain has the structure 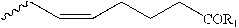
wherein R₁ is an alkyloxy or alkylamino group, preferably with 1-10 carbons, especially 1-6 carbons; and
the omega chain is defined by the formula 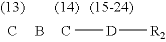
wherein B is a single bond or a double bond, C is a carbon atom, the number being indicated within parentheses, D is a carbon chain with 2-5, especially 3 carbon atoms, C₁₅ having a carbonyl or (S)—OH substituent and C₁₆-C₁₉ having lower alkyl substituents, or preferably H, and R₂ is a phenyl ring optionally having substituents selected among alkyl, alkoxy and fluorocarbon groups;
and
(b) a derivative of β-cyclodextrin having the structure 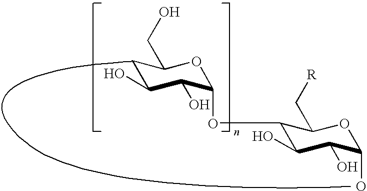
wherein n equals 6 and R is —NH₂⁺—(CH₂)ₚ—OH or —NH₂⁺—(CH₂)ₚ—NH₃⁺ (at acidic pH), p being an integer from 2-6.

2. The complex of claim 1 wherein in the omega chain of the prostaglandin derivative B is a single bond, D is a chain of 3 carbon atoms, and R₂ is a phenyl group.

3. The complex of claim 1 wherein the derivative of a prostaglandin is selected from the group consisting of 15-dehydro-17-phenyl-18,19,20-trinor-PGF_{2α}-isopropylester,13,14-dihydro-17-phenyl-18,19,20-trinor-PGA₂-isopropyl ester, 15-(R)-17-phenyl-18,19,20 trinor-PGF_{2α}, latanoprost, bimatoprost and travoprost.

4. The complex of claim 1 wherein the derivative of a prostaglandin is selected from the group consisting of latanoprost, bimatoprost and travoprost.

5. The complex of claim 1 wherein the derivative of a prostaglandin is latanoprost.

6. The complex of claim 1 wherein R of the derivative of β-cyclodextrin is —NH₂⁺—(CH₂)₃—NH₃⁺ or —NH₂⁺—(CH₂)₃—OH.

7. The complex of claim 1 wherein the derivative of a cyclodextrin and the derivative of a prostaglandin are present at a molar ratio from 1:1 to 30:1.

8. The complex of claim 1 wherein the derivative of a cyclodextrin and the derivative of a prostaglandin are present at a molar ratio from 5:1 to 10:1.

9. A pharmaceutical composition for topical treatment of ocular hypertension and glaucoma containing a non-covalent complex and an opthalmologically compatible vehicle, the non-covalent complex consisting of
(a) a derivative of a prostaglandin in an amount sufficient to reduce intraocular pressure, the prostaglandin derivative having the general structure 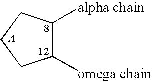
wherein A represents the alicyclic ring C₈-C₁₂ of PGA, PGB, PGD, PGE or PGF; the alpha chain has the structure 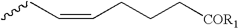
wherein R₁ is an alkyloxy or alkylamino group, preferably with 1-10 carbons, especially 1-6 carbons; and
the omega chain is defined by the formula 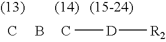
wherein B is a single bond or a double bond, C is a carbon atom, the number being indicated within parentheses, D is a carbon chain with 2-5, especially 3 carbon atoms, C₁₅ having a carbonyl or (S)—OH substituent and C₁₆-C₁₉ having lower alkyl substituents, or preferably H, and R₂ is a phenyl ring optionally having substituents selected among alkyl, alkoxy and fluorocarbon groups;
and
(b) a derivative of β-cyclodextrin having the structure 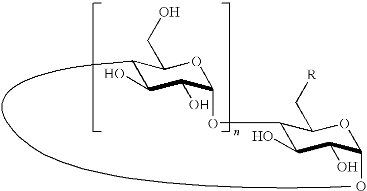
wherein n equals 6 and R is —NH₂⁺—(CH₂)ₚ—OH or —NH₂⁺—(CH₂)ₚ—NH₃⁺ (at acidic pH), p being an integer from 2-6.

10. The pharmaceutical composition of claim 9 wherein in the omega chain of the prostaglandin derivative B is a single bond, D is a chain of 3 carbon atoms, and R₂ is a phenyl group.

11. The pharmaceutical composition of claim 9 wherein the derivative of a prostaglandin is selected from the group consisting of 15-dehydro-17-phenyl-18,19,20-trinor-PGF_{2α}-isopropylester, 13,14-dihydro-17-phenyl-18,19,20-trinor-PGA₂-isopropylester, 15-(R)-17-phenyl-18,19,20-trinor-PGF_{2α}, latanoprost, bimatoprost and travoprost.

12. The pharmaceutical composition of claim 9 wherein the derivative of a prostaglandin is selected from the group consisting of latanoprost, bimatoprost and travoprost.

13. The pharmaceutical composition of claim 9 wherein the derivative of a prostaglandin is latanoprost.

14. The therapeutic composition of claim 9 wherein R of the derivative of β-cyclodextrin is —NH₂⁺—(CH₂)₃—NH₃⁺ or NH₂⁺—(CH₂)₃—OH.

15. The pharmaceutical composition of claim 9 further including a viscosity-increasing agent.

16. The pharmaceutical composition of claim 9 further including a preservative.

17. The pharmaceutical composition of claim 9 further including an anti-oxidant.

18. The pharmaceutical composition of claim 13 further including a viscosity-increasing agent.

19. The pharmaceutical composition of claim 13 further including a preservative.

20. The pharmaceutical composition of claim 13 further including an anti-oxidant.

21. A container comprising a pharmaceutical composition of claim 9 capable of dispensing the composition in a drop-wise fashion to an eye of a patient.

22. A kit comprising two or more containers of claim 21.

23. A container capable of dispensing a therapeutic composition in a drop-wise fashion to an eye of a patient, the container comprising two compartments, wherein a first compartment comprises a non-covalent complex according to claim 1 and a second compartment comprises an opthalmologically compatible vehicle, and wherein the two compartments are capable of being brought in communication such that a therapeutic composition is constituted.

24. A kit comprising two or more containers of claim 23.

25. A kit comprising a first container comprising a non-covalent complex according to claim 1 and a second container comprising an opthalmologically compatible vehicle.

26. A method of treating glaucoma or intraocular hypertension in an eye of a patient, comprising topical administration to the eye of the patient of a pharmaceutical composition of claim 9.
